(19) United States
(12) Patent Application Publication
(10) Pub. No.: US 2025/0049865 A1
(43) Pub. Date: Feb. 13, 2025

(54) MICROBIAL COMPOSITION WITH WEIGHT LOSS EFFECT AND APPLICATION THEREOF

(71) Applicant: SUZHOU PRECISION BIOTECH CO., LTD., Jiangsu (CN)

(72) Inventors: Yongliang ZHU, Jiangsu (CN); Xiaojing MU, Jiangsu (CN); Mengmeng ZHU, Jiangsu (CN); Qingqing XU, Jiangsu (CN)

(73) Assignee: SUZHOU PRECISION BIOTECH CO., LTD., Jiangsu (CN)

(21) Appl. No.: 18/722,595

(22) PCT Filed: Nov. 3, 2022
(86) PCT No.: PCT/CN2022/129525

§ 371 Date: Jun. 21, 2024

(30) Foreign Application Priority Data
Dec. 22, 2021 (CN) 202111580338.5

(51) Int. Cl. A61K 35/747; A61P 3/04

(52) U.S. Cl. CPC A61K 35/747; A61P 3/04

(57) ABSTRACT

The invention provides a microbial composition with weight loss efficacy, the microbial composition including Lactobacillus gasseri and Akkermansia muciniphila; a ratio of the Lactobacillus gasseri and the Akkermansia muciniphila is 2:3-3:2 by viable counts. By probiotic composition containing a specific proportion and content of Lactobacillus gasseri and Akkermansia muciniphila, it is able to improve body weight abnormalities caused by metabolic diseases.

### TECHNICAL FIELD

The present invention relates to the field of microorganisms, in particular to a microbial combination with weight loss efficacy and its application thereof.

### BACKGROUND OF RELATED ART

The types and quantities of microbial communities in the human gut are extremely large. The gut is not only the largest digestive organ in the body, but also an organ that integrates endocrine, immune, and barrier functions. When the physiological conditions are normal, the gut microbiota is in a dynamic equilibrium, they help the intestine digest and absorb food, and maintain normal metabolism of the body.

Lactobacillus gasseri is a species of the genus Lactobacillus, which is one of the most common bacteria in the human body. Studies have shown that Lactobacillus can reduce waist circumference, body mass index (BMI), and visceral fat, and has good weight loss effects; Akkermansia muciniphila (AKK) grows in the intestinal mucus layer and utilizes the mucin secreted by the host to competitively repel and protect the intestine from pathogens. Studies on humans and mice have repeatedly shown that compared to healthy individuals or rodents, overweight or obese subjects/animals have reduced AKK abundance in their bodies. However, there is relatively little research on the weight loss effects of AKK on subjects or animals, as well as how to enhance its efficacy.

The commonly used weight loss drugs in modern treatment of obesity include liraglutide, oxaliplatin, sibutramine, and rimonabant, etc. But many weight loss drugs are restricted by the market or withdrawn from clinical use because some of them cannot achieve the expected effects or cause adverse reactions in patients. Therefore, there is an urgent need to develop an effective method or drug with minimally effective for treating obesity and related diseases.

Chinese patent with application No. 201910941360.4 discloses a weight loss composition and its preparation method, wherein the raw materials for each weight loss composition include: 4-16 billion inactivated AKK, 2-10 billion Bacillus natto, 1-16 billion Lactobacillus reuteri, and 5 g 20 g prebiotics. The invention adopts multiple weight loss mechanisms to enhance the weight loss effect of the combination, overcoming the drawbacks of previous weight loss products with a single weight loss mechanism and poor weight loss effect; The weight loss composition provided by the invention has a small weight per serving, a good taste of the product, does not affect the daily dietary habits of users, and avoids the following phenomena that often occur when taking general weight loss products: malnutrition, weakened immunity, later rebound, and affecting normal metabolic function. But it involves many bacterial species and the preparation process is relatively complex.

Chinese patent with application No. 202110686051.4 discloses a method for preparing a slimming probiotic soft capsule. The solid material is added to an oil agent, thoroughly mixed, and the resulting content suspension is obtained. Then, colloidal grinding is used to remove bubbles and obtain the content solution. Glycerin, xylitol, and purified water are added to a sol jar, heated to 80° C., stirred, and then gelatin is added to remove bubbles. After passing through an 80 mesh sieve, the soft shell material of the capsule is obtained, and the soft capsule is pressed; Put the compressed soft capsules into a drying rotary cage for shaping and drying to obtain slimming probiotic soft capsules. However, the invention can be further optimized for strain formulation.

In addition, although there have been many reports on the use of AKK for weight loss, existing technology also indicates that the gut microbiota is involved in various diseases, such as colon cancer, depression, and obesity, indicating that fecal microbiota transplantation may cause changes in the recipient host microbiota, thereby putting them at greater risk in these situations. The present invention found that the role of AKK in animal obesity models is limited, and there are also some strains of probiotics that can have positive effects on the human body by interacting with other intestinal microorganisms. Based on this, the present invention has discovered a more effective probiotic combination containing AKK, solving the limitation of using AKK alone.

### SUMMARY OF THE INVENTION

To solve the above problems, the present invention provides a mixed microorganism that achieve a better therapeutic effect in weight loss through the combination of two probiotics.

On one hand, the present invention provides the application of Lactobacillus gasseri and Akkermansia muciniphila in the preparation of drugs for the treatment and/or prevention and/or adjunctive treatment of obesity and obesity complications.

The described obesity includes, but is not limited to, genetic obesity and acquired obesity.

The acquired obesity includes but is not limited to: obesity caused by high-fat diet, obesity caused by drugs, obesity caused by endocrine disorders, obesity caused by sleep deprivation, obesity caused by metabolic disorders, obesity caused by mental factors, or obesity caused by age.

Said obesity caused by the described psychiatric factors includes, but is not limited to: obesity caused by vagal excitation, or obesity caused by sympathetic excitation.

The complications of obesity described include: hypertension, hyperlipidemia, diabetes, fatty liver, gallstones, cholecystitis, hyperuricemia, osteoporosis, gout, coronary heart disease, myocardial infarction, cerebral infarction, stroke of one or more kinds.

The ratio of viable counts between Lactobacillus gasseri and Akkermansia muciniphila was 2:3-3:2 when being used.

Preferably, said the ratio of viable counts between Lactobacillus gasseri and Akkermansia muciniphila is 1:1 when being used.

Said Lactobacillus gasseri is the culture or offspring culture of Lactobacillus gasseri LG08, and said Akkermansia muciniphila is the culture or offspring culture of Akkermansia muciniphila with number CCTCC NO: M 2020950.

The described Akkermansia muciniphila CCTCC NO: M 2020950 was obtained from healthy adult feces, and the culture was kept in China on Jan. 5, 2021, at Wuhan University, China.

The number of live bacteria in the microbial composition includes 40-60 copies of Lactobacillus gasseri and 40-60 copies of Akkermansia muciniphila.

Preferably, the number of live bacteria in the microbial composition includes 50 copies of Lactobacillus gasseri and 50 copies of Akkermansia muciniphila.

On the other hand, the present invention provides a microbial composition for the aforementioned use.

Said microbial compositions include Lactobacillus gasseri and Akkermansia muciniphila.

The ratio of viable counts for Lactobacillus gasseri and Akkermansia muciniphila is 2:3-3:2.

Preferably, the ratio of viable counts between Lactobacillus gasseri and Akkermansia muciniphila is 1:1.

Preferably, the number of viable counts in the microbial composition includes 40-60 copies of Lactobacillus gasseri and 40-60 copies of Akkermansia muciniphila.

Further, preferably, the number of live bacteria in the microbial composition includes 50 copies of Lactobacillus gasseri, 50 copies of Akkermansia muciniphila.

Preferably, the Lactobacillus gasseri is derived from the culture or offspring culture of Lactobacillus gasseri LG08 of Jiangsu Wecare Biotechnology Co., Ltd. and can be grown by existing methods; the Akkermansia muciniphila is from a culture or offspring culture number CCTCC NO: M 2020950.

Said Akkermansia muciniphila CCTCC NO: M 2020950 was obtained from healthy adult feces, and the culture was kept in China on Jan. 5, 2021, at Wuhan University, China.

Said Lactobacillus gasseri and Akkermansia muciniphila can be in the form of bacterial powder or bacterial fluid upon application.

Preferably, the viable counts content in the bacterial solution is 1×10⁸-1×10¹⁰ CFU/mL, preferably 5×10⁸-5×10¹⁰ CFU/mL, and further preferably 1×10⁹ CFU/mL.

Preferably, the microbial composition in the application is lyophilized powder and the viable counts content is 1×10⁸-1×10¹¹ CFU/g; preferably 5×10⁸-5×10¹⁰ CFU/g and further preferably 1×10⁹ CFU/g.

On the other hand, the present invention provides the preparation method of the aforementioned microbial composition.

The preparation described include culture of Lactobacillus gasseri and culture of Akkermansia muciniphila.

The culture of Lactobacillus gasseri includes the following steps:
S1, activization of Lactobacillus gasseri strain;
S2, culture of Lactobacillus gasseri strain.

Preferably, the medium for the Lactobacillus gasseri strain is MRS medium; further preferably, the MRS medium is formulated below:

| Ingredient | Content (g/L) |
|---|---|
| Peptone | 10.0 |
| Beef powder | 8.0 |
| Yeast powder | 4.0 |
| Glucose | 20.0 |
| K₂HPO₄ | 2.0 |
| Ammonium citrate dibasic | 2.0 |
| NaAc | 5.0 |
| MgSO₄ | 0.2 |
| MnSO₄ | 0.04 |
| Tween 80 | 1.0 |

The culture of Akkermansia muciniphila consists of the following steps:
S1, activization of Akkermansia muciniphila strain;
S2, culture of Akkermansia muciniphila strain.

Preferably, the medium formulation of the Akkermansia muciniphila strain in culture is listed below:

| Ingredient | Content (/200 mL) | |
|---|---|---|
| Brain heart infusion | 5.92 | g |
| Mucin of pig stomach | 0.48 | g |
| L-Cysteine hydrochloride | 0.3 | g |
| Thr | 1.0 | g |
| Vitamin K | 0.2 | mL |
| N-Acetyl-D-Glucosamine | 1.0 | g |
| Trace elements | 0.1 | mL |
| Tween-80 | 0.1 | mL |

The preparation also includes mixing of Lactobacillus gasseri cultures and Akkermansia muciniphila cultures according to the ratio of 2:3-3:2 by number of alive bacterium.

On the other hand, the invention provides a bacterial agent.

The agent includes Lactobacillus gasseri and Akkermansia muciniphila.

The bacterial agent may be liquid or solid or semi-solid.

On the other hand, the present invention provides a method of preparing a drug for the treatment or prevention of obesity.

The preparation method includes the preparation method of the microbial composition.

On the other hand, the present invention provides a drug used for the treatment or prevention of obesity.

The drug includes the aforementioned microbial composition and/or bacterial agent.

The drug may include a bacterial powder or bacterial solution prepared from the aforementioned microbial composition.

Preferably, the viable counts content in the bacterial solution is 1×10⁸-1×10¹⁰ CFU/mL, preferably 5×10⁸-5×10¹⁰ CFU/mL, and further preferably 1×10⁹ CFU/mL.

Preferably, the bacterial powder is a lyophilized powder; the viable counts content of the lyophilized powder is 1×10⁸-1×10¹¹ CFU/g CFU/g; preferably 1×10⁹ CFU/g.

Other pharmaceutically acceptable carriers or excipients or diluents may also be included in the drug described.

On the other hand, the present invention provides a health care product, feed, food product, dietary supplement, nutritional supplement or food additive used for the treatment or prevention of obesity.

Said health care product, feed, food product, dietary supplement, nutritional supplement or food additive include the aforementioned microbial composition and/or bacterial liquid.

Said health care product, feed, food product, dietary supplement, nutritional supplement or food additive may include bacterial powder or bacterial liquid prepared by the aforementioned microbial composition.

Preferably, the viable counts content in the bacterial solution is 1×10⁸-1×10¹⁰ CFU/mL, preferably 5×10⁸-5×10¹⁰ CFU CFU/mL, and further preferably 1×10⁹ CFU/mL.

Preferably, the bacterial powder is a lyophilized powder; the viable counts content of the lyophilized powder is 1×10⁸-1×10¹¹ CFU/g; preferably 1×10⁹ CFU/g.

### Beneficial Effects of the Present Invention

The probiotic composition that containing a specific proportion and content of Lactobacillus gasseri and Akkermansia muciniphila can improve body weight abnormalities caused by metabolic diseases.

### BRIEF DESCRIPTION OF THE DRAWINGS

FIG. 1 shows the weight change of mice with probiotic single or mixed bacteria before the modeling and starting the invention.

FIG. 2 shows the rate of body weight change in mice after initiation of probiotic single or mixed bacteria in the invention.

### DETAILED DESCRIPTION OF THE EMBODIMENTS

The invention is further elaborated in combination with specific embodiments, which are not used to limit the invention, but only to describe the invention. The experimental methods used in the following embodiments are not specially specified, and usually the materials and reagents used in the following embodiments.

The probiotic mixture of the following embodiments can be combined in other proportions, in order to make the description concise, the combinations of each proportions in the above embodiments are not described, however, as long as the combination of these technical features is not contradictory, it should be considered to be the scope recorded in this specification. Meanwhile, the following is only a preferred embodiment of the invention and is not used to limit the invention to those skilled in the art. Any modification, equivalent substitution, improvement made within the spirit and principles of the invention shall be included within the scope of protection of the invention.

### Embodiment 1 Culture of Lactobacillus gasseri and Akkermansia muciniphila

This embodiment provides culture of Lactobacillus gasseri and Akkermansia muciniphila in the present invention.

(1) Culture of Lactobacillus gasseri

Activation of strain: Lactobacillus gasseri LG08 (Jiangsu Wecare Biotechnology Co., Ltd., China, oblique preservation) used in the present invention was inoculated into 50 mL MRS culture medium and incubated in 37° C. and 150 bottles for 24 h to obtain seed liquid.

Fermentation and culture: The activated seed solution was inoculated into 1 L MRS medium at 1% inoculum volume (volume ratio), and then incubated in 37° C. and 150 revolutions for 48 h.

The MRS medium is as below:

| Ingredient | Content (g/L) |
|---|---|
| Peptone | 10.0 |
| Beef powder | 8.0 |
| Yeast powder | 4.0 |
| Glucose | 20.0 |
| K₂HPO₄ | 2.0 |
| Ammonium citrate dibasic | 2.0 |
| NaAc | 5.0 |
| MgSO₄ | 0.2 |
| MnSO₄ | 0.04 |
| Tween 80 | 1.0 |

(2) Culture of Akkermansia muciniphila

Activation of strain: the Akkermansia muciniphila (CCTCC NO: M 2020950, oblique preservation) used in the invention was inoculated into 50 mL special medium in an anaerobic workstation, and 37° C. strict anaerobic culture for 72 h to obtain the seed liquid.

Fermentation and culture: the activated seed solution was inoculated into 1 L special culture medium according to 1% inoculum volume (volume ratio), and 37° C. was strictly anaerobic culture for 72-96 h.

The specific culture medium is formulated in the table below:

| Ingredient | Content (/200 mL) | |
|---|---|---|
| Brain heart infusion | 5.92 | g |
| Mucin of pig stomach | 0.48 | g |
| L-Cysteine hydrochloride | 0.3 | g |
| Thr | 1.0 | g |
| Vitamin K | 0.2 | mL |
| N-Acetyl-D-Glucosamine | 1.0 | g |
| Trace elements | 0.1 | mL |
| Tween-80 | 0.1 | mL |

Brain heart infusion was purchased from Hopebio Biotechnology Co., Ltd. (Tsingdao) under Art. No. HB8297-5.

Mucin of pig stomach was purchased from SIGMA under Art. No. M2378-100G.

The trace elements are formulated in the table below:

| Ingredient | Content (g/L) |
|---|---|
| ZnCl₂ | 0.07 |
| H₃BO₃ | 0.06 |
| CuCl₂•2H₂O | 0.015 |
| FeCl₂•4H₂O | 1.5 |
| Na₂MoO₄•2H₂O | 0.025 |
| NiCl₂•6H₂O | 0.025 |
| MnCl₂•4H₂O | 0.1 |
| CoCl₂•6H₂O | 0.12 |

### Embodiment 2 Mouse Model Assay

An in vivo test of the monobacteria and microbial mixture in the implementation Embodiment 1 in the high-fat diet-induced obesity mouse model for the treatment or prevention of obesity, which aim at comparing the efficacy of single bacteria and mixture.

### Empirical Method:

(1) After centrifuge the bacterial solution and discard the supernatant of Lactobacillus gasseri and Akkermansia muciniphila individually, the concentration was adjusted to 1×10⁹ CFU/mL with sterile PBS for each, and the mice were gavaged according to the following groups:
Lactobacillus gasseri alone;
Akkermansia muciniphila alone;
Lactobacillus gasseri and Akkermansia muciniphila 1:1 ratio mix;
the total gastric volume was 0.2 mL once daily in each group.

(2) Animal experiment: 30 numbers of 10-week-old C57BL/6 female mice (purchased from JunKe Biological Co., Ltd., Nanjing, China) were purchased and fed in the same environment. 6 mice were fed a normal diet, and 24 mice were fed high-fat diet (60% fat calories, 15% protein calories, and 25% carbohydrate calories, purchased from Trophic Animal Feed High-tech Co., Ltd, Nantong. China, feed code: TP 2330055AC) for 8 weeks. The diet induced obesity model was established with a weight of 28.00±2.00 g. After successful modeling, the mice were divided into groups as follows:
Group 1: Normal maintenance feed+sterile physiological saline;
Group 2: High-fat feed+sterile physiological saline;
Group 3: High-fat feed+Lactobacillus gasseri (with a live bacterial count of 1×10⁹ CFU/mL, 0.2 mL per mouse orally administered);
Group 4: High-fat feed+mucin Akkermansia muciniphila (with a live bacterial count of 1×10⁹ CFU/mL, 0.2 mL per mouse orally administered);
Group 5: A combination of high-fat feed, Lactobacillus gasseri, and Akkermansia muciniphila (with a live bacterial count of 1×10⁹ CFU/mL and a bacterial ratio of 1:1).

Among them, the fat calorie of normal maintenance feed is 12%, while of the high-fat feed is 60%.

Administer continuously for 30 days, and measure the weight of mice every 2 days.

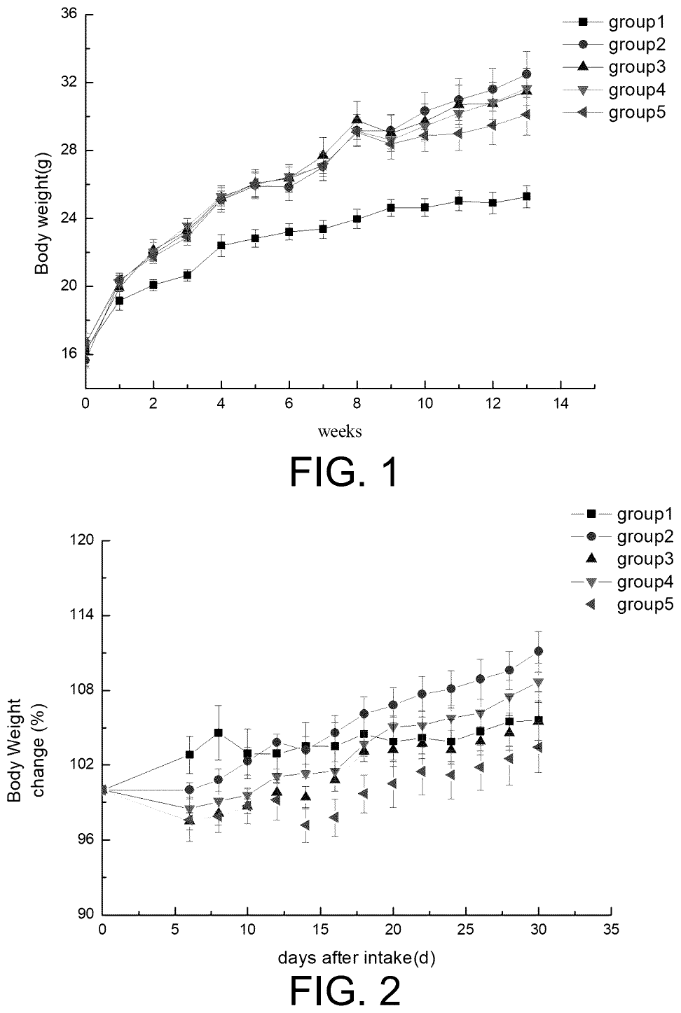

The experimental results are shown in FIG. 1-FIG. 2. FIG. 1 shows the weekly body weight change of each group of mice throughout the entire experimental process (modeling experiment for the first 8 weeks and within 4 weeks of starting administration). FIG. 2 shows the body weight change ratio of each group of mice after successful modeling and administration. Obesity model induction was conducted in the first 8 weeks, and after grouping according to body weight in the 8th week, medication was administered in groups 1-5. According to FIG. 1, it can be seen that after starting administration, group 5 mice showed a significant weight loss compared to group 2. After 4 weeks of administration, the weight loss of mice was about 8%. However, there was almost no difference between group 3 and group 4, and there was no significant difference compared to group 2. Combined with FIG. 2, group 5 mice showed the most significant weight loss. The results showed that single bacteria could slightly reduce the weight of obese mice, but the effect was not significant, while mixed microorganisms could effectively reduce the weight of obese model mice.

The results show that the probiotic mixture provided by the embodiments of the present invention can be widely used in preparing drugs, food or health care products by regulating the intestinal barrier function.

### Contratest

Referring to the method in Embodiment 2, the Contratest is set. The difference is that after successful modeling, in addition to high-fat feed, the following feeding methods are also used:

| | The difference from the group 5 | |
|---|---|---|
| | in Embodiment 2 | Results |
| Contratest 1 | Live bacteria ratio: | After administration (Week 13), the |
| | Lactobacillus gasseri and | experimental group had significantly lower |
| | Akkermansia muciniphila 1:5 | weight than the control (no dose) mice, |
| | | resulting in close to group4. |
| Contratest 2 | Live bacteria ratio: | After administration (Week 13), the |
| | Lactobacillus gasseri and | experimental group had significantly lower |
| | Akkermansia muciniphila 5:1 | weight than the control (no dose) mice, |
| | | resulting in close to group3. |
| Contratest 3 | Concentration of single | After the end of the administration (Week |
| | bacterial | 13), there was no significant difference |
| | solution1 × 10⁷ CFU/mL | between the body weight of the |
| | | experimental group and the control group |
| | | (no administration), indicating that the |
| | | concentration of bacterial fluid was not |
| | | effective in weight loss. |
| Contratest 4 | Concentration of single | After the end of the administration (Week |
| | bacterial | 13), there was no significant difference |
| | solution1 × 10¹¹ CFU/mL | between the body weight and the mice of |
| | | 1109 CFU/mL. If the high concentration of |
| | | the bacterial solution did not strengthen the |
| | | efficacy, some mice developed loose stools. |

1. A microbial composition comprising Lactobacillus gasseri and Akkermansia muciniphila; wherein a ratio of the Lactobacillus gasseri and the Akkermansia muciniphila is 2:3-3:2 by viable counts.

2. The microbial composition according to claim 1, wherein the ratio of the Lactobacillus gasseri and the Akkermansia muciniphila is 1:1 by viable counts.

3. The microbial composition according to claim 1, wherein the Lactobacillus gasseri is a culture or offspring culture of Lactobacillus gasseri LG08, and the Akkermansia muciniphila is a culture or offspring culture of Akkermansia muciniphila having a deposit number of CCTCC NO: M 2020950.

4. Application of the microbial composition according to claim 1 in the preparation of drugs for the treatment and/or prevention and/or assisting in the treatment of obesity and its complications.

5. The application according to claim 4, wherein the obesity comprises hereditary obesity and acquired obesity.

6. The application according to claim 4, wherein the obesity complications include one or more of hypertension, hyperlipidemia, diabetes, fatty liver, gallstones, cholecystitis, hyperuricemia, osteoporosis, gout, coronary heart disease, myocardial infarction, cerebral infarction, and stroke.

7. A microbial agent, characterized in that, comprising the microbial composition according to claim 1.

8. A drug for treating and/or preventing and/or assisting in the treatment of obesity and its complications, wherein the drug comprises the microbial composition according to claim 1.

9. Application of the microbial composition according to claim 1 in the preparation of health products, feed, food product, dietary supplements, nutritional supplements, or food additives for maintaining body shape.

10. A product using the health products, feed, food product, dietary supplements, nutritional supplements, or food additives according to claim 9, comprising Lactobacillus gasseri and Akkermansia muciniphila.

11. The application according to claim 4, wherein the acquired obesity includes: obesity caused by a high-fat diet, obesity caused by medication, obesity caused by endocrine disorders, obesity caused by insufficient sleep, obesity caused by metabolic disorders, obesity caused by psychological factors, or obesity caused by aging.

12. The application according to claim 11, wherein the obesity caused by psychological factors includes obesity caused by vagus nerve stimulation, or obesity caused by sympathetic nerve stimulation.

13. A drug for treating and/or preventing and/or assisting in the treatment of obesity and its complications, wherein the drug comprises the bacterial agent according to claim 7.
